(19) United States
(12) Patent Application Publication
(10) Pub. No.: US 2026/0235511 A1
(43) Pub. Date: Aug. 13, 2026

(54) ANALYSIS DEVICE FOR ANALYZING WORKING LIQUIDS

(71) Applicant: LIFE ELETTRONICA SOCIETA'A RESPONSABILITA' LIMITATA, Modena (MO) (IT)

(72) Inventors: Riccardo GALLI, Modena (MO) (IT); Luigi ROVATI, Modena (MO) (IT); Valentina DI PINTO, Modena (MO) (IT)

(73) Assignee: LIFE ELETTRONICA S.r.l., Modena (MO) (IT)

(21) Appl. No.: 19/471,155

(22) PCT Filed: Apr. 2, 2024
(86) PCT No.: PCT/IB2024/053171

§ 371 Date: Sep. 30, 2025

(30) Foreign Application Priority Data
Mar. 30, 2023 (IT) 102023000006222

(51) Int. Cl. G01N 21/51; G01N 21/17; G01N 21/53; G01N 21/64; G01N 21/85

(52) U.S. Cl. CPC G01N 21/51; G01N 21/532; G01N 21/645; G01N 21/6486; G01N 21/85; G01N 2021/174; G01N 2021/6491

(57) ABSTRACT

An analysis device comprising at least one container body within which one working liquid is contained; at least one emitter; and at least one detecting assembly, arranged outside the container body and configured to detect at least one output electromagnetic radiation. The detecting assembly comprises at least two different detectors selected from the list comprising: at least one absorption detector configured to detect at least one transmitted output electromagnetic radiation defined by the input electromagnetic radiation transmitted through the working liquid; at least one fluorescence detector configured to detect at least one generated output electromagnetic radiation defined by the endogenous fluorescence generated by the working liquid as a result of the exposure to the input electromagnetic radiation; at least one scattering detector configured to detect at least one scattered output electromagnetic radiation defined by the input electromagnetic radiation scattered by the working liquid.

### TECHNICAL FIELD

The present invention relates to an analysis device for analyzing working liquids.

### BACKGROUND ART

Some analysis devices are known for analyzing working liquids employed to verify one or more characteristics of interest of at least one working liquid, such as e.g. its composition or similar characteristics.

This type of devices generally makes use of one or more detectors configured to detect the influence of the working liquid on the behavior of an appropriate electromagnetic radiation.

In fact, the working liquid interacts with the electromagnetic radiation differently according to its own characteristics.

In this way, the devices of this type make it possible to detect certain characteristics of the working liquid.

However, this kind of devices are susceptible to some refinements.

In fact, the need to detect the presence of different molecules and/or substances within the same working liquid is well known.

Various molecules and/or substances, however, interact with electromagnetic radiation in substantially similar ways, and therefore it is difficult to distinguish their presence in the working liquid.

This drawback makes the use of this type of device particularly limited. In particular, this drawback makes it particularly inconvenient to use this type of device for particularly sensitive applications such as the detection of toxins in the blood, e.g., in blood processing systems.

In fact, the need to distinguish different molecules and/or substances in the blood easily and quickly is particularly felt in these systems.

### DESCRIPTION OF THE INVENTION

The main aim of the present invention is to devise an analysis device for analyzing working liquids which allows a working liquid to be analyzed in a quick and easy manner.

A further object of the present invention is to devise an analysis device for analyzing working liquids which allows different molecules and/or substances in a working liquid to be distinguished in a quick and easy manner.

An additional object of the present invention is to devise an analysis device for analyzing working liquids which allows different molecules and/or substances in the blood to be detected in a quick and easy manner.

Still one object of the present invention is to devise an analysis apparatus for analyzing working liquids which allows different molecules and/or substances in the blood to be detected in a quick and easy manner.

Another object of the present invention is to devise an analysis device for analyzing working liquids which allows the aforementioned drawbacks of the prior art to be overcome within the framework of a simple, rational, easy and effective to use as well as cost-effective solution.

The aforementioned objects are achieved by this device having the characteristics of claim 1.

The aforementioned objects are achieved by this apparatus having the characteristics of claim 28.

### BRIEF DESCRIPTION OF THE DRAWINGS

Other characteristics and advantages of the present invention will become more apparent from the description of a preferred, but not exclusive, embodiment of an analysis device for analyzing working liquids, illustrated by way of an indicative, yet non-limiting example, in the accompanying tables of drawings in which:

FIG. 1 is a schematic view of the device according to the invention;

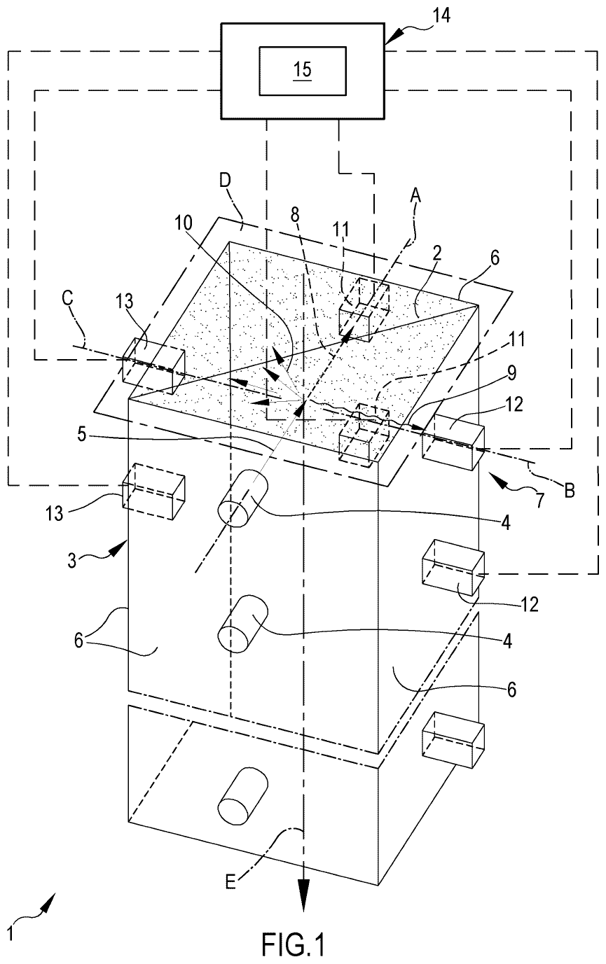

FIG. 2 is a schematic cross-sectional view of a possible, but not exclusive, embodiment of the device according to the invention;

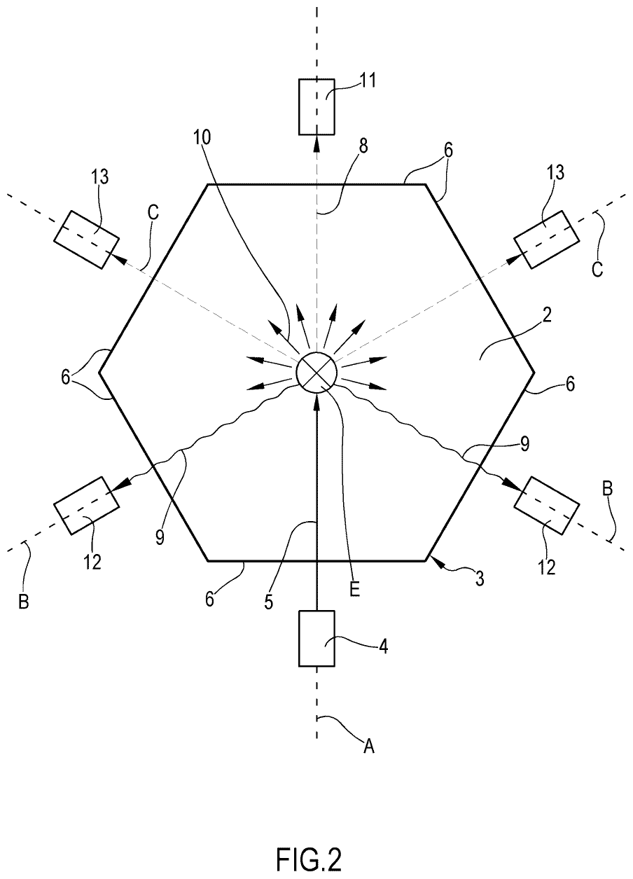

FIG. 3 is a schematic view of the apparatus according to the invention;

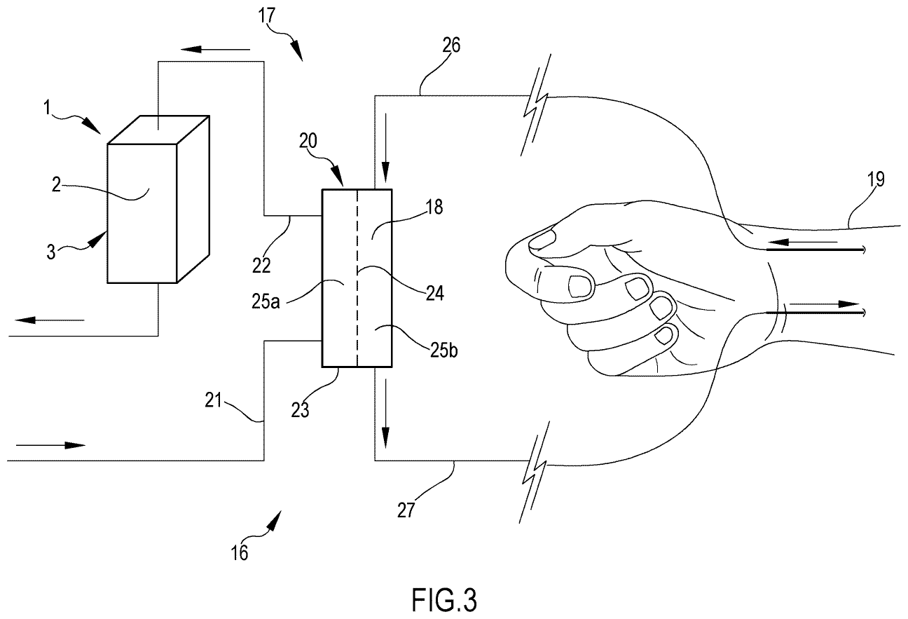

FIG. 4 is a schematic view of a possible, but not exclusive embodiment of the apparatus according to the invention.

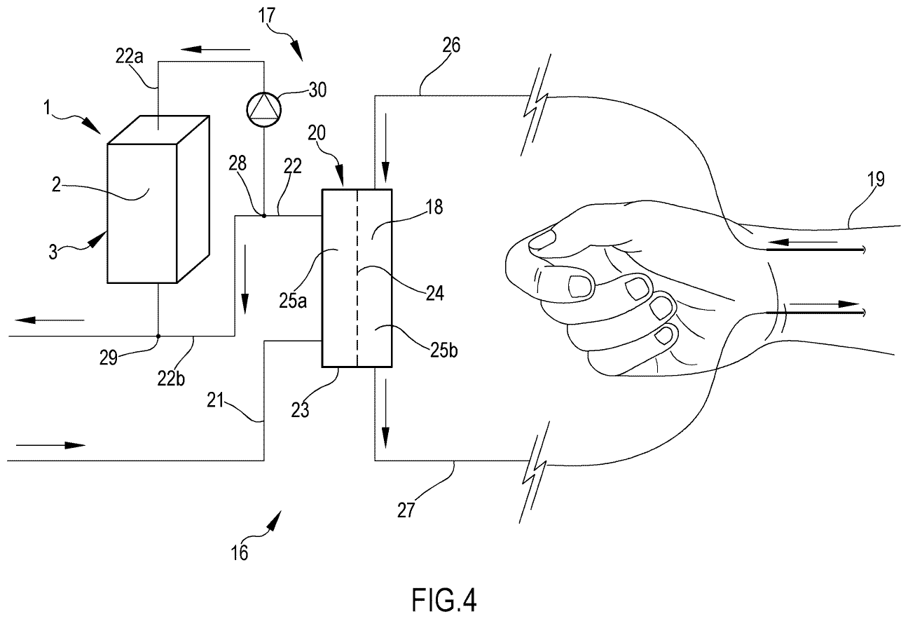

### EMBODIMENTS OF THE INVENTION

With particular reference to these figures, reference numeral 1 globally denotes an analysis device for analyzing working liquids.

The analysis device 1 for analyzing working liquids comprises at least one container body 3 within which at least one working liquid 2 to be analyzed is contained.

Preferably, the working liquid 2 flows, e.g., forcibly or naturally, along the container body 3 along a direction of flowing E.

Indeed, it cannot be ruled out that the container body 3 may be a transit body open on opposite sides, e.g. arranged along a line of flowing of the working liquid 2 and through which the working liquid flows along the direction of flowing E.

It cannot, however, be ruled out that the container body 3 holds the working liquid 2 within it, substantially in a state of stillness.

In addition, the device 1 comprises at least one emitter 4 arranged outside the container body 3 and configured to emit, substantially along a direction of propagation A, at least one input electromagnetic radiation 5 within the container body 3 for the analysis of the working liquid.

Appropriately, the direction of propagation A is arranged transversely, preferably substantially orthogonally, to the direction of flowing E.

Preferably, the emitter 4 is configured to emit an input electromagnetic radiation 5 having a wavelength of between 100 and 480 nm.

This wavelength is particularly adapted to detect the presence of uremic toxins in the working liquid 2, such as urea, uric acid, creatine, β2-microglobulin and the like. Preferably, the emitter 4 is configured to emit one input electromagnetic radiation 5 having as its wavelength that of maximum absorption of the uremic toxin that is to be detected, i.e., the concentration of which, e.g., is to be measured.

Below is table 1 comprising urea toxins detectable by the device 1 within the working liquid 2.

TABLE 1

| | PM | | PM | | PM |
|---|---|---|---|---|---|
| BASSO P.M. | (daltons) | PROTEIN BOUND | (daltons) | MEDIE MOLECOLE | (daltons) |
| ADMA mg/L | 202 | CMPF mg/L | 240 | Peptide natriuretico | 3.080 |
| | | | | atriale ng/L | |
| Acido β guanidino | 131 | Fruttoselina mg/L | 308 | β2 microglobulina mg/L | 11.818 |
| propionico mg/L | | | | | |
| Creatinina mg/L | 113 | Gliossalato μg/L | 58 | Cistatina C mg/L | 13.300 |
| Acido γ guanidino | 145 | Omocisteina ng/L | 145 | Interleuchina-1 βng/L | 32.000 |
| butirrico μg/L | | | | | |
| Ipoxantina mg/L | 136 | Indossil solfato mg/L | 241 | Interleuchina-6 ng/L | 24.500 |
| Mioinositolo mg/L | 180 | Leptina mg/L | 16.000 | Catene leggere k mg/L | 25.000 |
| Acido orotico mg/L | 174 | P-Cresolo mg/L | 108 | Catene leggere λmg/L | 45.000 |
| Ossalato mg/L | 90 | Pentosidina μg/L | 342 | Leptina μg/L | 16.000 |
| Urea g/L | 60 | Fenolo mg/L | 94 | Paratormone μg/L | 9.225 |
| Acido urico mg/L | 168 | Acido ippurico mg/L | 195 | Fattore D Complemento | 23.750 |
| | | | | mg/L | |
| Timina mg/L | 126 | Retinol binding protein | 21.200 | Retinol binding protein | 21.200 |
| | | mg/L | | mg/L | |
| Xantina mg/L | 152 | Spermidina μg/L | 145 | Tumor necrosis factor- | 26.000 |
| | | | | αng/L | |

Preferably, the emitter 4 is an electronic device for the emission of one or more electromagnetic radiations, such as e.g. an LED or the like.

Conveniently, the working liquid 2 is a dialyzing liquid.

According to the invention, the container body 3 is at least partly transparent to the input electromagnetic radiation 5 emitted by the emitter 4 so as to allow the exposure of the working liquid 2 to the same input electromagnetic radiation.

Preferably, the container body 3 is totally transparent to the input electromagnetic radiation 5 emitted by the emitter 4.

Preferably, the container body 3 comprises one or more lateral walls 6 containing the working liquid 2 having a substantially flat conformation. Furthermore, the direction of propagation A is preferably orthogonal to at least one lateral wall 6 facing which the emitter 4 is arranged.

Specifically, the device 1 comprises at least one detecting assembly 7, arranged outside the container body 3 and configured to detect at least one output electromagnetic radiation 8, 9, 10 out of the container body 3 as a result of the exposure of the working liquid 2 to the input electromagnetic radiation 5.

In more detail, the working liquid 2 affects the input electromagnetic radiation 5, by generating one or more of the output electromagnetic radiations 8, 9, 10 detected by the detecting assembly 7.

Even more in detail, the characteristics of one or more of the output electromagnetic radiations 8, 9, 10 differ depending on the characteristics of the working liquid 2 exposed to the input electromagnetic radiation 5.

According to the invention, the detecting assembly 7 comprises at least two different detectors 11, 12, 13 selected from the list comprising:
at least one absorption detector 11 configured to detect at least one transmitted output electromagnetic radiation 8 defined by the input electromagnetic radiation 5 transmitted through the working liquid 2;
at least one fluorescence detector 12 configured to detect at least one generated output electromagnetic radiation 9 defined by the endogenous fluorescence generated by the working liquid 2 as a result of the exposure to the input electromagnetic radiation 5;
at least one scattering detector 13 configured to detect at least one scattered output electromagnetic radiation 10 defined by the input electromagnetic radiation 5 scattered by the working liquid 2.

Preferably, the detectors 11, 12, 13 are electronic devices configured to detect electromagnetic radiations, such as photodiodes or the like.

For example, the absorption detector 11 is of the type of a photodiode, spectrometer or the like, the fluorescence detector 12 is of the type of a photodiode, APD, photomultiplier, spectrometer or the like, and the scattering detector 13 is of the type of a photodiode, APD, photomultiplier or the like.

Preferably, the fluorescence detector 12 is configured to detect a generated output electromagnetic radiation 9 having a wavelength of between 490 and 1000 nm.

In other words, the detecting assembly 7 comprises at least two different detectors 11, 12, 13, that is, configured to detect different output electromagnetic radiations 8, 9, 10. In this way, the device 1 allows the working liquid 2 to be analyzed based on at least two different output electromagnetic radiations 8, 9, 10, thus optimizing the analysis of the working liquid 2.

For example, this expedient allows the device 1 to detect within the working liquid two different molecules and/or substances that are difficult to distinguish using the analysis devices of known type.

Preferably, the detecting assembly 7 comprises at least one absorption detector 11, at least one fluorescence detector 12 and at least one scattering detector 13.

In this way, the device 1 allows the working liquid 2 to be analyzed on the basis of at least three different output electromagnetic radiations 8, 9, 10, further optimizing the analysis of the working liquid 2.

Advantageously, the absorption detector 11 and/or the fluorescence detector 12 are adapted to detect the presence of specific molecules and/or substances within the working liquid 2.

In particular, the absorption detector 11 and/or the fluorescence detector 12 are adapted to detect the presence of uremic toxins in the working liquid 2, such as e.g. urea, uric acid, creatine, β2-microglobulin and the like.

Conveniently, the scattering detector 13 is configured to check for particulate matter in the working liquid 2, e.g. red blood cells or the like.

Advantageously, the absorption detector 11 is arranged substantially opposite the emitter 4 substantially along the direction of propagation A.

Preferably, the container body 3 comprises a plurality of lateral walls 6.

Advantageously, the emitter 4 and the absorption detector 11 are arranged on opposite sides of the container body 3 where respective lateral walls 6 are arranged substantially parallel and opposite each other. Thus, preferably, the direction of propagation A is substantially orthogonal to the lateral walls 6 facing the emitter 4 and the absorption detector 11.

Conveniently, the fluorescence detector 12 is arranged along a direction of generation B arranged substantially transversely with respect to the direction of propagation A.

Appropriately, the direction of generation B is arranged transversely, preferably substantially orthogonally, to the direction of flowing E.

Preferably, the direction of generation B is arranged substantially orthogonal to the direction of propagation A.

Appropriately, the direction of generation B is arranged substantially orthogonal to at least one lateral wall 6 of the container body 3 facing which the fluorescence detector 12 is arranged.

In particular, the direction of generation B is arranged substantially orthogonally to at least one lateral wall 6 which is different from the lateral wall 6 to which the direction of propagation A is orthogonal.

Conveniently, the scattering detector 13 is arranged along a direction of scattering C arranged substantially transverse with respect to the direction of propagation A.

Appropriately, the direction of scattering C is arranged transversely, preferably substantially orthogonally, to the direction of flowing E.

Preferably, the direction of scattering C is arranged orthogonally to the direction of propagation A.

Appropriately, the direction of scattering C is arranged substantially orthogonally to at least one lateral wall 6 of the container body 3 facing which the scattering detector 13 is arranged.

In particular, the direction of propagation C is arranged substantially orthogonal to at least one lateral wall 6 which is different from the lateral wall 6 to which the direction of propagation A is orthogonal.

Preferably, the emitter 4, the absorption detector 11, the fluorescence detector 12 and the scattering detector 13 are arranged facing lateral walls 6 which are different from each other.

Preferably, the fluorescence detector 12 and the scattering detector 13 are arranged on opposite sides of the container body 3 where respective lateral walls 6 arranged substantially parallel and opposite each other are located.

Advantageously, the direction of propagation A and at least one of either the direction of generation B and the direction of scattering C lie on the same plane of detection D. Preferably, the direction of propagation A, the direction of generation B and the direction of scattering C lie on the same plane of detection D.

Appropriately, the direction of flowing E is transverse, preferably substantially orthogonal, to the plane of detection D.

Advantageously, the lateral walls 6 define a container body 3 having a polygonal cross section.

Preferably, the container body 3 has a polygonal cross section with mirrored lateral walls 6.

It cannot be ruled out that the detecting assembly 7 comprises a plurality of fluorescence detectors 12.

Preferably, the fluorescence detectors 12 of the same detecting assembly 7 are arranged facing lateral walls 6 which are different from each other.

It cannot be ruled out that the detecting assembly 7 may comprise a plurality of scattering detectors 13.

Preferably, the scattering detectors 13 of the same detecting assembly 7 are arranged facing lateral walls 6 which are different from each other.

Conveniently, the device 1 comprises processing means 14 configured to process at least one datum indicative of the concentration of at least one or more molecules and/or substances contained within the working liquid 2 depending on one or more output electromagnetic radiations 8, 9, 10 detected by the detecting assembly 7.

Specifically, the processing means 14 are configured to process a plurality of indicative data.

Preferably, the term indicative datum means any measured and/or processed digital datum which is indicative, directly or indirectly, of the concentration of at least one or more molecules and/or substances contained within the working liquid 2 depending on one or more output electromagnetic radiations 8, 9, 10 detected by the detecting assembly 7.

Preferably, the processing means 14 are configured to process the indicative datum substantially in real time.

It cannot be ruled out that the processing means 14 may be configured to process the indicative datum substantially continuously and/or periodically over time.

Conveniently, the processing means 14 are configured to process the indicative datum during the movement of the working liquid 2 along the direction of flowing E.

Preferably, the processing means 14 are of the type of an electronic data processing system, such as e.g. a computer, server or the like.

Advantageously, the processing means 14 comprise at least one artificial intelligence system 15 configured to perform machine learning and/or deep learning methods to process the indicative data.

Conveniently, the device 1 comprises a plurality of emitters 4, each configured to emit an input electromagnetic radiation 5 having a different wavelength.

In addition, the device 1 comprises a plurality of detecting assemblies 7, each configured to detect at least one output electromagnetic radiation 8, 9, 10 out of the container body 3 as a result of the exposure of the working liquid 2 to the input electromagnetic radiation 5 emitted by a corresponding emitter 4.

In other words, the device 1 comprises at least one detecting assembly 7 for each emitter 4.

Specifically, each wavelength is selected appropriately to detect one or more specific characteristics of the working liquid 2, e.g. to detect the presence of one or more specific molecules and/or substances.

Preferably, the detecting assemblies 7 comprise the same number and the same type of detectors 11, 12, 13.

Further embodiments of the device 1 cannot however be ruled out, wherein one or more detecting assemblies 7 may comprise a different number of detectors 11, 12, 13 and/or detectors of different type than the other detecting assemblies 7.

Conveniently, the processing means 14 are configured to process at least one indicative datum depending on one or more of the output electromagnetic radiations 8, 9, 10 detected by a corresponding detecting assembly 7.

In other words, the processing means 14 are configured to process at least one indicative datum for each detecting assembly 7.

Preferably, the processing means 14 are configured to process a plurality of indicative data for each detecting assembly 7.

### Conveniently:

the emitters 4 are spaced away from each other along the container body 3; and/or
the detecting assemblies 7 are spaced away from each other along the container body 3.

Preferably, the emitters 4 are aligned with each other and/or the detecting assemblies 7 are aligned with each other.

Conveniently, each emitter 4 together with the corresponding detecting assembly 7 defines a relevant plane of detection D.

Preferably, the planes of detection D are arranged parallel to each other.

According to a further aspect, the present invention relates to the use of an analysis device 1 for analyzing working liquids of the type of dialyzing liquids employed by a treatment system 16 of the blood 18, preferably of the type of a dialysis system, e.g. of the hemodialysis type.

Preferably, the device 1 described with reference to its use comprises one or more of the characteristics described above with reference to the device 1.

According to a further aspect, the present invention relates to an analysis apparatus 17 for analyzing working liquids for the treatment of the blood 18 of a patient 19, comprising:
treatment means 20 of the blood 18, adapted to treat the blood 18 coming from a patient 19 with at least one working liquid 2, the working liquid 2 treating the blood 18 as a result of the treatment means 20;
at least a first conveyance duct 21 adapted to convey the working liquid 2 for the treatment of the blood 18 at inlet to the treatment means 20;
at least a second conveyance duct 22 adapted to convey the working liquid 2 that has treated the blood 18 at output from the treatment means 20;
at least one analysis device 1, the container body 3 being connected to the second conveyance duct 22 to receive the working liquid 2 that has treated the blood 18.

Preferably, the container body 3 is arranged along the second conveyance duct 22. In this way, the working liquid 2 flowing along the second duct 22 flows through the container body 3.

Advantageously, the absorption detector 11 and/or the fluorescence detector 12 are adapted to detect the presence of specific molecules and/or substances within the working liquid 2 that has treated the blood 18.

In particular, the absorption detector 11 and/or the fluorescence detector 12 are adapted to detect the presence of uremic toxins in the working liquid 2, such as e.g. urea, uric acid, creatine, β2-microglobulin and the like.

Conveniently, the scattering detector 13 is configured to check for the presence of particulate matter in the working liquid 2, such as e.g. red blood cells or the like and/or such as foreign bodies traceable, e.g. to the membrane rupture introduced later in this disclosure.

Specifically, the working liquid 2 interacts with and treats the blood 18 as a result of the treatment means 20.

In more detail, the working liquid 2 is adapted to receive one or more molecules and/or substances from the blood 18 as a result of the treatment means 20.

In even more detail, the working liquid 2 is adapted to receive one or more molecules and/or substances that are toxic to the patient.

In this way, the working liquid 2 treats the blood 18 of the patient by purifying it of one or more molecules and/or substances.

In fact, the working liquid 2 that has treated the blood 18 through the treatment means 20, i.e., the working liquid 2 flowing into the second conveyance duct 22, contains within it the molecules and/or substances received from the blood 18.

In this way, the device 1 analyzes the working liquid 2 that has treated the blood 18 and allows the concentration of one or more molecules and/or substances received from the blood 18 of the patient to be checked.

Advantageously, the treatment means 20 comprise a dialyzer 23 and the working liquid 2 is a dialyzing liquid.

Preferably, the dialyzer 23 comprises a semi-permeable membrane 24 that divides the dialyzer itself substantially into two chambers 25a, 25b, where one chamber 25a receives the working liquid 2 and the other chamber 25b receives the blood 18 of the patient. Advantageously, the scattering detector 13 allows the conditions of the membrane 24 to be checked, e.g., by detecting in the working liquid 2 the presence of one or more foreign bodies received from the blood 18 as a result of a malfunction of the membrane 24 and/or traceable to the membrane itself.

Conveniently, through the membrane 24 (by processes known in the field) the working liquid 2 receives one or more of the molecules and/or substances contained in the blood 18, by treating it.

Specifically, the working liquid 2 for treatment is fed into the corresponding chamber 25a by means of the first duct 21, while the working liquid 2 that has treated the blood 18 exits the same chamber 25a through the second duct 22.

In this way, the working liquid 2 that has treated the blood 18 reaches the container body 3 to be analyzed.

Specifically, the container body 3 is connected to the second duct 22 in a fluid-operated manner so that at least part of the liquid received from the latter flows along the direction of flowing E.

Appropriately, the apparatus 17 comprises at least one extracorporeal flowing line 26, 27 of the blood 18 provided with:
at least one delivery stretch 26 adapted to convey the blood 18 to be treated at output from the patient 19 and at inlet to the treatment means 20; and/or
at least one return stretch 27 adapted to convey the treated blood 18 at output from the treatment means 20 and at inlet to the patient 19.

Specifically, the blood 18 to be treated is fed into the corresponding chamber 25b via the delivery stretch 26, while the treated blood 18 flows out of the same chamber 25b via the return stretch 27.

Appropriately, the flowing line 26, 27 is an extracorporeal flowing line of the type known in the dialysis system industry.

Preferably, the device 1 described with reference to the apparatus 17 comprises one or more of the characteristics described above with reference to the device 1 and/or to its use.

Advantageously, the second duct 22 comprises at least one branch point 28 where it divides into at least two branches 22a, 22b, each adapted to receive a part of the working liquid 2 that has treated the blood 18.

Specifically, the device 1 is connected along one of the branches 22a, 22b. It cannot, however, be ruled out that the apparatus 17 may comprise a plurality of devices 1, each connected along a different branch 22a, 22b.

Preferably, the second duct 22 divides into one analysis branch 22a along which the device 1 is arranged and into at least one exhaust branch 22b.

Conveniently, the second duct 22 comprises at least one junction point 29 where the branches 22a, 22b rejoin.

Specifically, the device 1 is connected along one of the branches 22a, 22b between the branch point 28 and the junction point 29.

In other words, the branch 22a along which the device 1 is arranged is a bypass branch.

It cannot be ruled out that the apparatus 17 may comprise pumping means 30 adapted to forcibly convey part of the working liquid 2 that has treated the blood 18 into the branch 22a along which the device 1 is arranged.

Alternatively, it cannot be ruled out that the working liquid 2 that has treated the blood 18 may naturally flow within the branches 22a, 22b.

According to a further aspect, the present invention relates to the use of an apparatus 17 for the treatment of the blood 18 of a patient 19 in a treatment system 16 of the blood 18, preferably of the type of a dialysis system, e.g. of the hemodialysis type.

Preferably, the apparatus 17 described with reference to its use comprises one or more of the characteristics described above with reference to the apparatus 17.

According to a further aspect, the present invention relates to a treatment system 16 of the blood 18 comprising at least one of either the device 1 or the apparatus 17.

Preferably, the device 1 and/or the apparatus 17 described with reference to the treatment system 16 comprise one or more of the characteristics of the device 1 and of the apparatus 17 described with reference to the device 1 and/or to its use and to the apparatus 17 and/or to its use, respectively.

It has in practice been ascertained that the described invention achieves the intended objects.

In particular, the fact is emphasized that the measuring assembly allows analyzing a working liquid in an easy and quick manner.

In addition, the detecting assembly allows distinguishing different molecules and/or substances in the working liquid in an easy and quick manner.

In particular, the processing means allow processing data on the concentration of such molecules and/or substances in the working liquid.

In more detail, such processing is further optimized by means of the artificial intelligence system.

Advantageously, the detecting assembly makes the device particularly suitable for detecting different molecules and/or substances in the blood in an easy and quick manner.

1. An analysis device for analyzing working liquids, the analysis device comprising:
at least one container body within which at least one working liquid to be analyzed is contained;
at least one emitter, arranged outside said container body and configured to emit, substantially along a direction of propagation, at least one input electromagnetic radiation within said container body for the analysis of the working liquid, said container body being at least partly transparent to the input electromagnetic radiation emitted by said emitter so as to allow the exposure of the working liquid to the same input electromagnetic radiation; and
at least one detecting assembly, arranged outside said container body and configured to detect at least one output electromagnetic radiation out of said container body as a result of the exposure of the working liquid to said input electromagnetic radiation; wherein
said detecting assembly comprises at least two different detectors selected from the list comprising:
at least one absorption detector configured to detect at least one transmitted output electromagnetic radiation defined by said input electromagnetic radiation transmitted through the working liquid;
at least one fluorescence detector configured to detect at least one generated output electromagnetic radiation defined by the endogenous fluorescence generated by the working liquid as a result of the exposure to said input electromagnetic radiation;
at least one scattering detector configured to detect at least one scattered output electromagnetic radiation defined by said input electromagnetic radiation scattered by the working liquid.

2. The analysis device according to claim 1, wherein said emitter is configured to emit said input electromagnetic radiation having a wavelength of between 100 and 480 nm.

3. The analysis device according to claim 1, wherein said absorption detector is arranged substantially opposite said emitter substantially along said direction of propagation.

4. The analysis device according to claim 1, wherein said fluorescence detector is arranged along a direction of generation arranged substantially transverse to said direction of propagation.

5. The analysis device according to claim 1, wherein said fluorescence detector is configured to detect a generated output electromagnetic radiation having a wavelength of between 490 and 1000 nm.

6. The analysis device according to claim 1, wherein said scattering detector is arranged along a direction of scattering arranged substantially transverse to said direction of propagation.

7. (canceled)

8. The analysis device according to claim 1, wherein the working liquid flows along said container body, along a direction of flowing, and said container body is a transit body open on opposite sides and through which the working liquid flows along said direction of flowing.

9. The analysis device according to claim 8, wherein said direction of propagation is arranged orthogonal to said direction of flowing.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The analysis device according to claim 1, further comprising:
a plurality of emitters, each configured to emit said input electromagnetic radiation having a different wavelength; and
a plurality of detecting assemblies, each configured to detect at least one output electromagnetic radiation out of said container body as a result of the exposure of the working liquid to the input electromagnetic radiation emitted by a corresponding emitter.

22. The analysis device according to claim 21, further comprising: processing means, operationally connected to said detecting assembly and configured to process at least one datum indicative of the concentration of at least one or more molecules and/or substances contained within the working liquid depending on one or more output electromagnetic radiations detected by said detecting assembly.

23. The analysis device according to claim 22, wherein said processing means comprise at least one artificial intelligence system configured to perform machine learning and/or deep learning methods to process said indicative data.

24. The analysis device according to claim 22, wherein said processing means are configured to process said indicative datum substantially in real time.

25. The analysis device according to claim 22, wherein said processing means are configured to process said indicative datum substantially continuously and/or periodically over time.

26. (canceled)

27. (canceled)

28. An analysis apparatus for analyzing said working liquids for treatment of blood of a patient, the analysis apparatus comprising:
treatment means of the blood, adapted to treat the blood coming from said patient with said at least one working liquid, the working liquid treating the blood as a result of said treatment means;
at least a first conveyance duct adapted to convey the working liquid for the treatment of the blood at inlet to said treatment means;
at least a second conveyance duct adapted to convey the working liquid that has treated the blood at output from said treatment means; and
at least one of said analysis device according to claim 1, said container body being connected to said second conveyance duct to receive the working liquid that has treated the blood.

29. The analysis apparatus according to claim 28, wherein said treatment means comprise a dialyzer, wherein the working liquid is a dialyzing liquid.

30. The analysis apparatus according to claim 27, further comprising: at least one extracorporeal flowing fact line of the blood provided with:
at least one delivery stretch adapted to convey the blood to be treated at output from the patient and at inlet to said treatment means; and/or
at least one return stretch adapted to convey the treated blood at output from the treatment means and at inlet to the patient.

31. The analysis apparatus according to claim 28, wherein said second duct comprises at least one branch point where said second duct divides into at least two branches, each adapted to receive a part of the working liquid that has treated blood, said analysis device being connected along one of said branches.

32. The analysis apparatus according to claim 31, wherein said second duct comprises at least one junction point where said branches rejoin, said analysis device being connected along one of said branches between said branch point and said junction point.

33. The analysis apparatus according to claim 32, further comprising: pumping means adapted to forcibly convey part of the working liquid that has treated the blood into a branch of said branches along which the analysis device is arranged.

34. The analysis apparatus according to claim 32, wherein the working liquid that has treated the blood may naturally flow within said branches.
